(19) United States
(12) Patent Application Publication
(10) Pub. No.: US 2013/0110229 A1
(43) Pub. Date: May 2, 2013

(54) AORTAL TRICUSPID HEART VALVE PROSTHESIS

(71) Applicants: Leo Antonovich Bokeriya, Moscow (RU); Andrey Vasilevich Agafonov, Moscow (RU); Gennady Iraklievich Kiknadze, Moscow (RU); Alexandr Yurievich Gorodkov, Moscow (RU)

(72) Inventors: Leo Antonovich Bokeriya, Moscow (RU); Andrey Vasilevich Agafonov, Moscow (RU); Gennady Iraklievich Kiknadze, Moscow (RU); Alexandr Yurievich Gorodkov, Moscow (RU)

(21) Appl. No.: 13/695,782

(22) PCT Filed: May 11, 2010
(86) PCT No.: PCT/RU2010/000237

§ 371 Date: Jan. 15, 2013

(30) Foreign Application Priority Data
Apr. 30, 2010 (RU) 2010117033

(51) Int. Cl. A61F 2/24

(52) U.S. Cl. CPC A61F 2/24
USPC 623/2.19

(57) ABSTRACT

The technical result of the invention comprises maintaining the physiological structure of the flow of blood, both in the open and in the closed state of a valve on account of the construction and shape of the cusps. The technical result is achieved in that the heart valve prosthesis comprises a body with a flange, and a closing unit in the form of three cusps mounted in the through opening in the body, wherein each of the cusps has a central rib directed from the centre of the through section of the valve to the body, and convex-concave wings are arranged along the sides of the rib, and cusps are mounted in the body by means of a mounting unit, wherein, in the closed position, the geometry of the valve reproduces the geometry of an actual aortal valve.

### FIELD OF THE INVENTION

The invention relates to medical technology and can be used in cardiosurgery in operations to replace cardiac valves.

### BACKGROUND OF THE INVENTION

Description of the blood flow structure and determination of the physiological blood circulation norm are among the central problems of physiology, clinical pathophysiology, cardiology, and cardiosurgery.

Early results of studies into the structural organization of blood flow were obtained by high-speed blood flow filming by methods visualizing movement of roentgenopaque substances introduced into the bloodstream. It was established that the bloodstream has flow lines frequently corresponding to a spiral and practically has no stream mixing as blood flows through the central parts—the heart and great vessels—of the cardiovascular system. It was also demonstrated that normal blood flow is not turbulent, that it has a thin boundary layer on the walls of the flow canal, and has low energy dissipation along the flow.

After direct methods of blood velocity measurement in a stream (electromagnetic flow meters, film heat loss anemometers, and pulse ultrasonic Doppler velocimeters) were developed, it has been found that both the small thickness of the boundary layer and the complex profile shape of longitudinal velocities in the heart and aorta provide no evidence for this flow to be definitely considered laminar or turbulent.

Morphological studies and physical modeling, and cine-angiography and ventriculography methods used in the late 1970s contributed to establishing that:
asymmetric junction of the main cavities in the mainstream part of the cardiovascular system contributes to stream swirling;
some of the intracardiac trabeculas have a spiral orientation;
the swirl of the blood flow in the central blood circulation parts has a fragmented visualization; and
the nuclei of the endothelial cells in the aorta have a spiral orientation corresponding to the direction in which shear stresses are applied.

It was assumed, on the basis of these facts, that blood flows in the central parts of the blood circulation system in the form of a swirled stream, but direct attempts at visualizing and determining the structure of a real or simulated flow were unsuccessful

Development of new methods for studying liquid flows (MR-tomography and MR-velocimetry, color Doppler echocardiography, and laser anemometry) has created opportunities for three-dimensional measurement of the velocity field in the blood flow. In particular, color Doppler echocardiography has revealed blood flow swirling in the aorta, and MR-velocimetry has registered episodes of axisymmetric blood flow in the heart and several large arteries. These studies, too, have failed to provide a quantified description of the swirled blood flow because of no analytical or numerical methods being available for modeling flow in a canal of complex geometric configuration such as a bloodstream.

Still, a series of devices have been developed, on the basis of empirical observations, for use in cardiosurgery, which are intended to swirl the flow as a way for improving their functional characteristics.

The prosthetic heart valve of U.S. Pat. No. 5,207,707, A, May 4, 1993, comprises an annular body having three pivoted flat leaflets provided therein. This design is disadvantageous because of considerable load applied to the leaflet turning structure, for which reason the reliability and service life of the valve are reduced.

In another prosthetic cardiac valve (Patent WO 0038595, A1, Jul. 6, 2000), one of its alternative designs comprises an annular body having two rims of different thickness and a locking element having three cusps. One of the surfaces of each cusp is flat and the other, concave and spherical, the concave surface facing the steam flowing through the valve thereby causing serious disturbances in the natural structure of the bloodstream flowing through the valve. A further disadvantage of the prior art device is instability because of the small moment of force applied to each cusp upon the opening thereof.

A further prosthetic cardiac valve has an annular body and three flat pivotal cusps (Patent RU 2173969 C1, Sep. 27, 2001). Three cantilevered projections are provided on the inside surface of the body to secure the cusps thereto.

The prior are prosthetic valve is disadvantageous because the shape of the cusps does not reproduce the physiological structure of the blood flow, and besides, stagnation zones stimulating formation of thrombi may occur under the cantilevered projection during valve operation.

### SUMMARY OF THE INVENTION

A quantitative analysis of blood circulation in general and the structure of the bloodstream in the human heart and great vessels, as also this invention, could only be made after accurate solutions of non-stationary hydrodynamic equations for viscous fluids had been obtained (Kiknadze G. I., Krasnov Yu. K., “Evolution of a Spout-Like Flow of a Viscous Fluid,” Sov. Phys. Dokl., 1986; 31(10): 799-801). These solutions enabled a blood flow in the human heart and great vessels to be described in sufficiently full detail.

This invention is aimed at developing a prosthetic cardiac valve design that corrects the deficiencies of existing similar devices and has a high reliability and service life.

The practical result achieved by the use of the claimed device is based on the established fact that the blood flow in the blood circulation system of humans and animals is a swirled stream. The field of velocities and pressure field in the stream have been found by applying the accurate solutions of non-stationary equations of viscous fluid hydrodynamics (see: the cited article by Kiknadze G. I., Krasnov Yu. K., “Evolution of a Spout-Like Flow of a Viscous Fluid,” Sov. Phys. Dokl. 1986; 31(10)). Furthermore, formation and regulation mechanisms of a swirled stream in the heart and great vessels have been uncovered as well (Kiknadze G. I., Oleinikov V. G. Gachechiladze I. A., Gorodkov A. Yu., Dobrova N. B., Bakei Sh., and Bara Zh-L., “On the Structure of the Flow in the Left Ventricle of the Heart and Aorta Determined on the Basis of Accurate Solutions of Non-Stationary Equations in Hydrodynamics and Morphometric Studies,” Proceedings of the Academy of Sciences, Vol. 351, 1996, pp. 119-122); and the quantitative values of the principal parameters of a normal swirled bloodstream in the aorta of healthy volunteers have been calculated (Gorodkov A. Yu., Nikolaev D. A., “An Analysis of the Dynamic Characteristics of a Swirled Bloodstream by Measuring the Geometric Parameters of the Flow Canal by MR-Tomography,” Bulletin of the A.N. Bakulev Cardiovascular Surgery Science Center, Russian Academy of Medical Sciences, 2003, J4°9, pp, 67-69; Bokeria L. A., Gorodkov A. Yu., Kiknadze G. I., et al., “An Analysis of the Velocity Field of a Swirled Bloodstream in the Aorta on the Basis of 3D Mapping by MR-Velocimetry,” Bulletin of the A.N. Bakulev Cardiovascular Surgery Science Center, Russian Academy of Medical Sciences, 2003, No. 9, pp. 70-74). The results of recent studies have shown that accurate solutions of non-stationary hydrodynamic equations give an adequate picture of blood circulation in both the normal and pathological states and that flow circulation depending on the azimuthal velocity component is the principal compensatory factor influencing the heart remodeling process. In particular, the possibility of compensation of up to 90% of the mitral valve stenosis or up to 85% of regurgitation of the mitral valve by changing the azimuthal velocity component has been demonstrated (Bokeria L. A., Gorodkov A. Yu., Kiknadze G. I., Nikolaev D. A., Kliuchnikov, M. D. Alshibaya, “An Analysis of the Mechanisms of Compensation and Left Ventricle Remodeling upon Pathological Change of Cavity Geometry,” 11th Academic Session of the A.N. Bakulev Cardiovascular Surgery Science Center, Russian Academy of Medical Sciences, Moscow, May 13-15, 2007, Bulletin of the A.N. Bakulev Cardiovascular Surgery Science Center, Russian Academy of Medical Sciences, Vol. 8, No. 3, 2007: 200). Accurate solutions, therefore, help make an analysis of blood circulation in different conditions of the cardiovascular system.

The pattern of bloodstream swirling and mechanisms of bloodstream formation discovered were studied experimentally and theoretically and identified for the physiologically normal state of the organism. This was done by posthumous morphological measurements of the heart and aorta, dynamic reconstruction of the aorta by MR-tomography, measurement of the bloodstream velocity field in the aorta of healthy volunteers by MR-velocimetry, and a quantitative analysis of a swirled bloodstream by accurate Kiknadze-Krasnov solutions describing such flows.

The technical result achieved by the use of this invention consists in preservation of the physiological structure of the bloodstream in the open and closed positions of the valve owing to the design and shape of the cusps. The structural stream organization produced by the valve of this design helps attain a physiologically adequate blood flow without flow stagnation and separation zones.

The claimed technical result is achieved by providing the prosthetic cardiac valve that has a body with a rim and a locking unit in the form of three cusps placed in the through opening of the body, each of the cusps having a central rib directed away from the center of the through cross-section of the valve toward the body and convexo-concave blades being provided on the sides of the rib, the cusps being secured in the body by a fixing unit such that the geometry of the closed valve replicates the geometry of the natural aortal valve.

The body may be of annular design.

The fixing unit may be provided in the shape of a rectangular aperture at the base of the rib engaging a hook-shaped projection on the annular body.

### BRIEF DESCRIPTION OF THE DRAWINGS

FIG. 1 is a diametrical sectional view of the prosthetic cardiac valve with open cusps.

FIG. 2 is a top view of the prosthetic cardiac valve with open cusps.

FIG. 3 is a diametrical sectional view of the prosthetic cardiac valve with closed cusps.

FIG. 4 is a top view of the prosthetic cardiac valve with closed cusps.

FIG. 5 is a view of a cusp of the prosthetic cardiac valve.

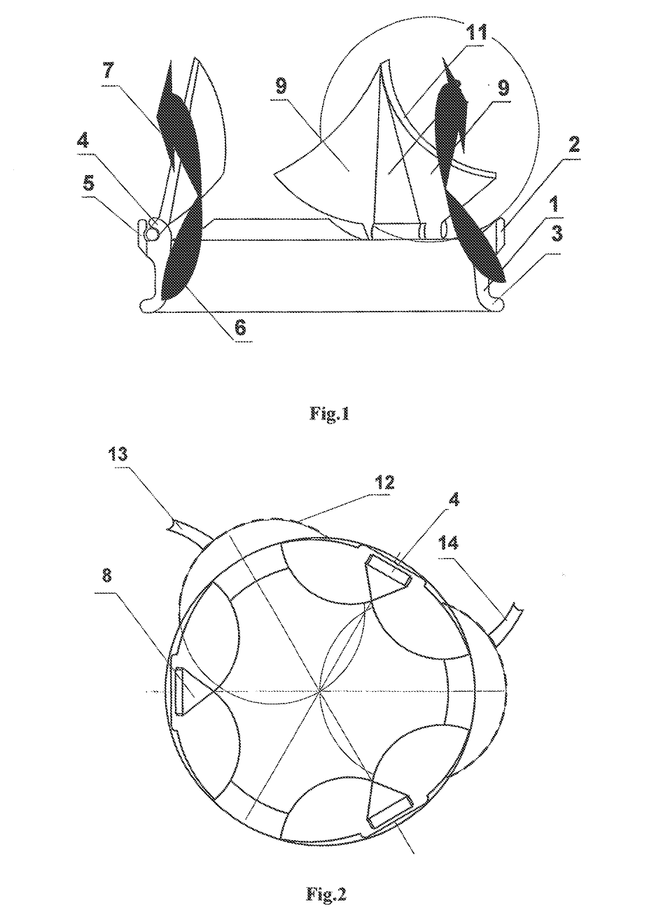

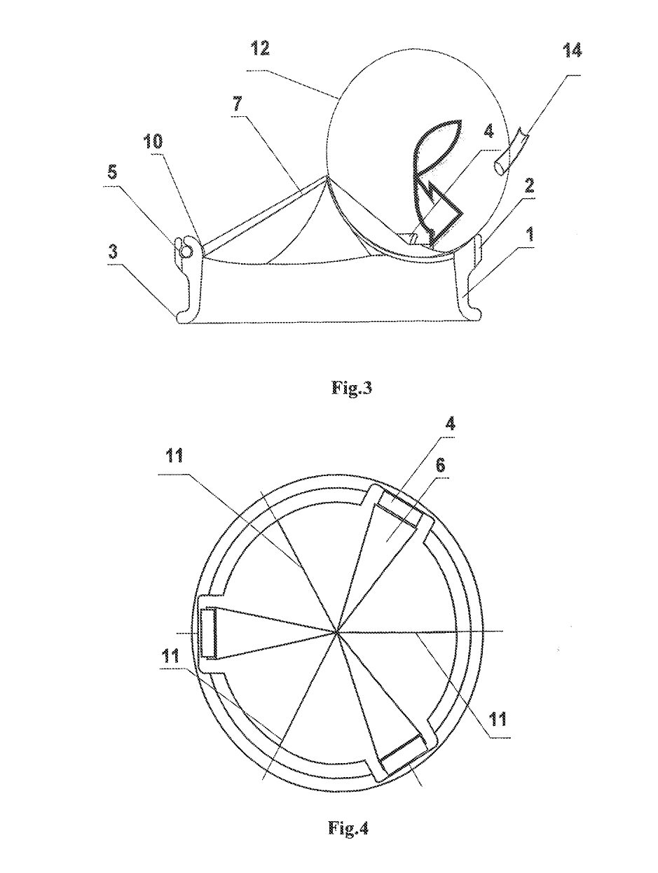

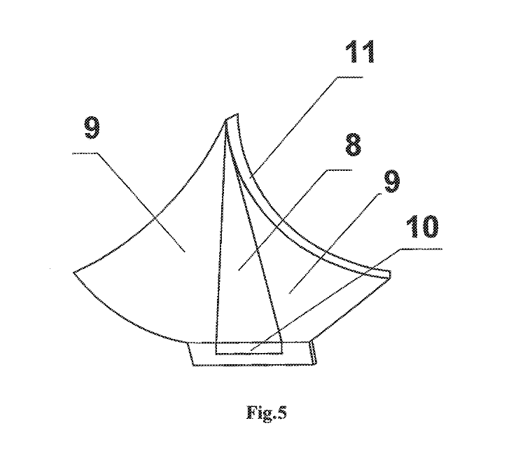

### EMBODIMENTS OF THE INVENTION

It has been demonstrated by morphological, functional, and experimental methods that the pulsating bloodstream in the heart and great vessels has a swirled structure so that blood flows without loss of energy and without forming flow separation and stagnation zones.

A quantitative analysis of the structure of the resulting bloodstream and the mechanisms of its generation and evolution in the heart and great vessels based on accurate solutions of non-stationary hydrodynamic equations for centripetal flows of a viscous fluid (Kiknadze G. I., Krasnov Yu. K., “Evolution of a Spout-Like Flow of a Viscous Fluid,” Sov. Phys. Dokl. 1986; 31(10): 799-801) made it possible, on the basis of experimental and clinical studies, to demonstrate conformity of the resultant accurate solutions to the common patterns of blood circulation physiology. The concept that was developed as a result harmonized the specifics of a swirled bloodstream and blood circulation function (Kiknadze G. I., Oleinikov V. G., Gachechiladze I. A., Gorodkov A. Yu., Dobrova N. B., Bakei Sh., and Bara Zh.-L., “On the Structure of the Flow in the Left Ventricle of the Heart and Aorta Determined on the Basis of Accurate Solutions of Non-Stationary Equations in Hydrodynamics and Morphometric Studies,” Proceedings of the Academy of Sciences, Vol. 351, 1996, pp. 119-122; and Gorodkov A. Yu., “An Analysis of the Structure of the Intracardiac Swirled Bloodstream on the Basis of the Morphometry of the Trabecular Relief of the Left Ventricle of the Heart,” Bulletin of the A.N. Bakulev Cardiovascular Surgery Science Center, Russian Academy of Medical Sciences, 2003, J b9, pp. 63-66).

The modern understanding of the nature of the swirled bloodstream in the heart, aorta, and great vessels is that, as was said above, the swirled stream formed at the outlet of the heart and evolving through heart contraction under the effect of the intracardiac trabecular relief is the principal component of the bloodstream in normal physiological conditions. The progress of a swirled stream in the blood flow canal is caused by steady localized reverse and secondary flows, and the resultant stream flows in the aorta without producing separation and stagnation zones and maintains physiologically normal blood distribution to regional pools. Further, the resultant stream combines all the swirled currents described by formulas (2.1 to 2.3) similarly to the dominant swirled stream expelled from the heart at a result of cardiac contraction.

Depending on the shape and spatial orientation of the trabeculas and papillary muscles, a swirled bloodstream is formed in cardiac chambers in the form of a spout having a radial velocity gradient C₀(t), blood circulating in a swirl Γₒ(i), and initial coordinate Z₀(t) in accordance with the accurate solutions of Kiknadze-Krasnov non-stationary hydrodynamic equations for swirled streams:

$$\begin{matrix}\text{?} = 2 {C}_{0} ( \text{?} ) \text{?} ( \text{?} ) + {C}_{1} ( \text{?} ) & ( 2.1 ) \\ \text{?} = - {C}_{0} ( \text{?} ) \text{?} ( \text{?} ) & ( 2.2 ) \\ \text{?} = \frac{{Γ}_{0} ( \text{?} )}{2 \text{?} ( \text{?} )} + \text{?} [ \frac{\text{?} ( \text{?} )}{2 \text{?} ( \text{?} )} ] \cdot [ Ψ [ ( \text{?} ( \text{?} ) + 1 ) \text{?} \text{?} ( \text{?} ) \text{?} ] ] \\ \text{?} \text{indicates text missing or illegible when filed} & ( 2.3 )\end{matrix}$$

wherein Vᵣ, V_{z}, V_{φ} are the radial, longitudinal, and tangential components of swirled current, r(t), z(t) and <p(t) are the cylindrical coordinates having the origin thereof combined with the sole point Z₀(t) within the stream canal where Vᵣ=V_{z}=V_{φ}=0, the position of said point changing over time as it moves along the stream;

C₀(t), Γ₀(i) and Γᵢ(t) are individual characteristics of the swirled stream, or the radial velocity gradient, main circulation, and multiple r-th circulations of the medium in the swirled streams generated in the composition of swirls combined by a single dominant circulation;

ψ[(Cᵢ+1), βᵢ(t)r²] is the incomplete Euler gamma function responsible for energy dissipation in a swirled current of viscous fluid controlling the process by an individual factor of each stream:

$$\text{?} ( t ) = \text{?} ( 0 ) exp \{ \frac{2 {\int }_{r} {C}_{0} ( τ ) d ( τ )}{1 - 4 v \text{?} ( 0 ) \text{?} d τ \cdot exp [ - 2 \text{?} \text{?} ( {τ}^{′} ) d {τ}^{′} ]} \}$$

$$\text{?} \text{indicates text missing or illegible when filed}$$

wherein V is kinematic viscosity of a medium involved in swirled current.

The present prosthetic cardiac calve comprises an annular body 1 having a lock ring 2 and a rim 3. Annular body 1 is provided with three hook-shaped projections 4 and a groove 5 opposite each of hook-shaped projections 4. The inner surface 6 of annular body 1 has a conical shape. Annular body 1 carries three cuspidate locking elements 7, each having a central rib 8 and two convexo-concave blades 9, a through rectangular slot being provided in the base of central rib 8. When the valve is closed cuspidate locking elements 7 close against one another along a closing line 11. The dotted line in the figure shows an approximate shape of aortal sinuses 12 and orifices of coronary arteries 13 and 14. The direction of the bloodstream is shown by arrows.

Cuspidate locking element 7 consists of a central rib 8 and two convexo-concave blades 9; it is a solid element made of pyrolytic carbon. Each of blades 9 replicates the shape of a closed aortal valve and is the surface of an ellipsis (⅛th part thereof).

The natural aortal valve consists of three flexible cusps placed in a configuration that may be described as a combination of three ellipses, ribs 8 thereof extending along the junction line of the ellipses and the blades replicating the free elliptical surface.

The valve operates as follows:

During the systolic period (when pressure in the left cardiac ventricle rises), cuspidate locking elements 7 turn in the direction of blood flow and rectangular groove 10 slides over the surface of hook-shaped projection 4 into contact with lock ring 2, and the cuspidate locking elements in the open state thereof shape the flow canal geometry such that it forms a minimum obstruction to the swirled bloodstream flowing out of the left ventricle. During the diastolic period (when pressure in the left ventricle is below the pressure in the aorta), cuspidate locking elements 7 turn in the opposite direction until the valve is fully closed. After the valve has been closed, the surface of the cuspidate locking elements reproduces authentically the geometry of the natural root of the aorta.

The stream structure is organized by the claimed valve to produce a physiologically adequate blood current free form stagnation and flow separation zones.

### INDUSTRIAL APPLICABILITY

The invention may be used for designing devices to replace organs in cardiovascular surgery (prosthetic cardiac valves) that restore adequate geometry of a patient's bloodstream and have improved safety by forming a swirled bloodstream structure. The present invention may be used in cardiosurgery to replace cardiac valves.

What is claimed is:

1. A prosthetic cardiac valve comprising a body having a rim and a locking unit in the form of three cusps arranged in the through opening of the body, each of the cusps having a central rib directed away from the center of the flow section of the valve to the body, and convexo-concave blades being provided at the sides of the rib, the cusps being secured in the body by a fixing unit, the geometry of the valve in the closed position thereof reproducing the geometry of the natural aortal valve.

2. The prosthetic cardiac valve of claim 1, wherein the body has an annular shape.

3. The prosthetic cardiac valve of claim 2, wherein the fixing unit is in the shape of rectangular orifice at the base of the rib to engage a hook-shaped projection provided on the annular body.
